(19) United States
(12) Patent Application Publication
(10) Pub. No.: US 2015/0343394 A1
(43) Pub. Date: Dec. 3, 2015

(54) HOLLOW FIBER MEMBRANE MODULE, METHOD FOR PRODUCING HOLLOW FIBER MEMBRANE, AND METHOD FOR PRODUCING HOLLOW FIBER MEMBRANE MODULE

(71) Applicant: TORAY INDUSTRIES, INC., Chuo-ku, Tokyo (JP)

(72) Inventors: Akihiro HAYASHI, Otsu-shi (JP); Masahiro OSABE, Otsu-shi (JP); Yoshiyuki UENO, Otsu-shi (JP)

(73) Assignee: TORAY Industries, Inc., Tokyo (JP)

(21) Appl. No.: 14/649,679

(22) PCT Filed: Feb. 13, 2014
(86) PCT No.: PCT/JP2014/053295

§ 371 Date: Jun. 4, 2015

(30) Foreign Application Priority Data
Feb. 20, 2013 (JP) 2013-030776

(51) Int. Cl. B01D 71/68; B01D 69/08; A61M 1/34; B01D 67/00; A61M 1/16; B01D 63/02; B01D 69/02

(52) U.S. Cl. CPC B01D 71/68; B01D 63/02; B01D 69/085; B01D 69/02; B01D 67/002; B01D 63/021; A61M 1/1621; A61M 1/3496; B01D 2325/38; B01D 2325/36; B01D 2323/42

(57) ABSTRACT

An object of the present invention is to provide a dry-type hollow fiber membrane module which is excellent in blood compatibility and elutes little eluted substance, and a hollow fiber membrane built in the module, and a method for producing a hollow fiber membrane module. Disclosed is a hollow fiber membrane module including a built-in hollow fiber membrane including a hydrophobic polymer and a hydrophilic group-containing polymer, the hollow fiber membrane module satisfying the following items: (a) the water content of the hollow fiber membrane is 10% by weight or less relative to the tare weight of the hollow fiber membrane, (b) the hydrophobic polymer contains no nitrogen, the hydrophilic group-containing polymer contains nitrogen, and the nitrogen content of the hollow fiber membrane is 0.05% by weight or more and 0.4% byweight or less, (c) the content of the hydrophilic group-containing polymer in the inner surface of the membrane is 20% by weight or more and 45% by weight or less, and (d) the consumption amount of an aqueous potassium permanganate solution (2.0×10⁻³ mol/L) used for titrating an eluted substance in 10 mL of a last part of a priming liquid is 0.2 mL or less per 1 m² of a membrane area.

### TECHNICAL FIELD

The present invention relates to a hollow fiber membrane module including a built-in hollow fiber membrane, which is excellent in blood compatibility and has low water content, and also elutes little eluted substance, and also relates to a method for producing the hollow fiber membrane and the hollow fiber membrane module.

### BACKGROUND ART

In recent years, a substance has been frequently separated by a hollow fiber membrane module including a built-in hollow fiber membrane. For example, an artificial kidney used in hemodialysis, a plasma separator used in plasmapheresis, and the like are exemplified.

Examples of the hollow fiber membrane module include a wet-type one in which a container is filled with a liquid and a hollow fiber membrane is completely filled with a liquid; a semi-dry-type one in which only a hollow fiber membrane is wetted, although a container is not filled with a liquid; and a dry-type one in which a hollow fiber membrane scarcely contains water. Of these, the dry-type one has advantages such as light weight and little possibility of deterioration of performance due to freezing even in cold districts, because of containing no water.

A high performance-type hollow fiber membrane having a large pore size is mainly used as a hollow fiber membrane used in a hollow fiber membrane module for blood processing, and it is capable of removing multiple pathogenic proteins having a medium/large molecular weight, such as β₂-microglobulin, and a hydrophobic polymer is mainly used as a membrane material. However, the hydrophobic polymer has poor blood compatibility because of its amplitude of hydrophobicity. Therefore, the addition of a hydrophilic component causes hydrophilization of a membrane surface, leading to improved blood compatibility.

However, if the hydrophobic component is exposed on a surface in contact with blood, when blood comes into contact with the hydrophobic component, there is a fear that activation of blood may cause proceeding of blood coagulation. Therefore, it can be said to be a preferable hollow fiber membrane if a surface thereof is uniformly coated with'the hydrophilic component.

A method for the addition of a hydrophilic component is generally a method in which a hydrophilic component is added to a membrane forming stock solution of a hollow fiber membrane, or a method in which a hollow membrane thus formed is immersed in a solution containing a hydrophilic component, thereby bonding the hydrophilic component. An efficient method for the addition of a hydrophilic component to a hydrophobic polymer includes a method for the addition of a hydrophilic group-containing polymer having a hydrophobic group as a constituent. An interaction between a hydrophobic group contained in the hydrophilic group-containing polymer and the hydrophobic polymer of a membrane material enhances introduction efficiency, thus enabling hydrophilization in an efficient manner.

Patent Literatures 1 and 2 disclose a dry-type hollow fiber membrane module including polysulfone as a hydrophobic polymer, and polyvinylpyrrolidone having a hydrophilic group (hereinafter abbreviated to PVP), which has low water content such as 0.2 to 7% by weight and elutes little eluted substance, and a method for producing the same. In order to realize reduction in an eluted substance, solution means of this method is to severely control the oxygen concentration by charging an oxygen scavenger in a packaging container, followed by irradiation with radiation.

Patent Literatures 3 and 4 disclose a method in which affinity with a hollow fiber membrane made of a hydrophobic polymer is enhanced using a copolymer composed of a hydrophobic group (hydrophobic unit) and a hydrophilic group (hydrophilic unit), thereby hydrophilizing an inner surface of a hollow fiber membrane in an efficient manner, and also mention a method in which a vinylpyrrolidone/vinyl acetate copolymer as a hydrophilic group-containing polymer is added to an injection liquid, thereby hydrophilizing the inner surface.

Patent Literature 5 discloses a method in which an inner surface of a hollow fiber membrane is modified by using an injection liquid containing a hydrophobicity modifier and a surfactant when a hollow fiber membrane is formed.

### CITATION LIST

### Patent Literature

[Patent Literature 1]

International Publication WO 2006/016573
[Patent Literature 2]

International Publication WO 2006/068124
[Patent Literature 3]

International Publication WO 2009/123088
[Patent Literature 4]

Japanese Unexamined Patent Publication (Kokai) No. 2012-115743
[Patent Literature 5]

Japanese Unexamined Patent Publication (Kokai) No. 10-235171

### SUMMARY OF INVENTION

### Technical Problem

However, in Inventions mentioned in Patent Literatures 1 and 2, since the entire membrane has comparatively high PVP content, not only the concentration of oxygen in a packaging container, but also relative humidity in the packaging container and steam permeability of the packaging container must be controlled, actually, so as to realize low elution, and it is impossible to perform irradiation with radiation until the concentration of oxygen sufficiently deceases, leading to a problem such as complicated production process.

Technologies mentioned in Patent Literatures 3 and 4 have not made a study of the optimum content of the hydrophilic group-containing polymer from the viewpoint of an eluted substance and blood compatibility in a dry-type module, and there is no mention of suppression of the eluted substance. Rather, there has hitherto been a trend of considering that sufficient amount of a hydrophilic component cannot be imparted to a hollow fiber membrane if the proportion of the polymer in an injection liquid must be increased when the hydrophilic group-containing polymer is allowed to contain in the injection liquid. The addition of an excess amount of the polymer may lead to an increase in elution amount.

There is a need for the method mentioned in Patent Literature 5 to remove a surfactant by washing with water, so that the shortage of water washing may lead to an increase in the amount of the eluted substance. The water content of a hollow fiber membrane is not also mentioned.

Therefore, an object of the present invention is to provide a dry-type hollow fiber membrane module which is excellent in blood compatibility and elutes little eluted substance, and a hollow fiber membrane built the module, and a method for producing a hollow fiber membrane module.

The inventors have intensively studied the above object and found that there is a possibility to achieve the object by using a method in which a hydrophilic group-containing polymer is added to an injection liquid when a hollow fiber membrane is formed, or a method in which a surface of a hollow fiber membrane is coated. with a hydrophilic group-containing polymer after forming a hollow fiber membrane.

Meanwhile, the inventors have also found that the above object cannot be achieved only by hydrophilizing a surface of a hollow fiber membrane using a hydrophilic group-containing polymer.

In other words, there has never been established technology to obtain a low water content hollow fiber membrane module, which suppresses elution of a substance from a hollow fiber membrane and is also excellent in blood compatibility, by controlling a state of a hydrophilic group-containing polymer of a surface of a hollow fiber membrane.

### Solution to Problem

The gist of the present invention lies in a hollow fiber membrane module including a built-in hollow fiber membrane including a hydrophobic polymer and a hydrophilic group-containing polymer, the hollow fiber membrane module satisfying the following items:
(a) the water content of the hollow fiber membrane is 10% by weight or less relative to the tare weight of the hollow fiber membrane,
(b) the hydrophobic polymer contains no nitrogen, the hydrophilic group-containing polymer contains nitrogen, and the nitrogen content of the hollow fiber membrane is 0.05% by weight or more and 0.4% by weight or less,
(c) the content of the hydrophilic group-containing polymer in the inner surface of the membrane is 20% by weight or more and 45% by weight or less; and
(d) the consumption amount of an aqueous potassium permanganate solution (2.0×10⁻³ mol/L) used for titrating an eluted substance in 10 mL of a last part of a priming liquid is 0.2 mL or less per 1 m² of a membrane area.

As mentioned in (a), the hollow fiber membrane module according to the present invention is expected to be a dry-type one, and enables low elution performance and high blood compatibility in a module including a built-in low water content hollow fiber membrane. As mentioned above, the hollow fiber membrane module includes a hydrophobic polymer and a hydrophilic group-containing polymer. As mentioned in (b), in order to use the nitrogen content as an index of the hydrophilic group content, the hollow fiber membrane module to be used is a module in which the hydrophobic polymer contains no nitrogen, while the hydrophilic group-containing polymer contains nitrogen (provided that at least one hydrophilic group-containing polymer may contain nitrogen when using two or more hydrophilic group-containing polymers). While an attempt is made to reduce elution by adjusting the nitrogen content to 0.05% by weight or more and 0.4% by weight at an optional position of the entire membrane, sufficiently high hydrophilicity is achieved by allowing an inner surface of a hollow fiber membrane to have 20% by weight or more and 45% by weight or less of hydrophilic groups, as mentioned in (c). Moreover, as mentioned in (d), the hollow fiber membrane module elutes little eluted substance and also has high blood compatibility.

Examples of the hydrophilic group-containing polymer include a hydrophilic polymer such as PVP, and also include a hydrophilic group-containing polymer having a hydrophobic group. The latter preferably has an ester group. Anyway, the polymer preferably has a pyrrolidone group, and it is also possible to use a copolymer of vinyl acetate with vinylpyrrolidone.

The present invention is characterized in that a hollow fiber membrane is obtained by using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.

Irradiation with radiation is preferably performed in a state where the water content of the hollow fiber membrane is adjusted to 10% by weight or less relative to the tare weight of the hollow fiber membrane built in the module.

Thus, the present invention adopts the following constitutions.
[1]

A hollow fiber membrane module including a built-in hollow fiber membrane including a hydrophobic polymer and a hydrophilic. group-containing polymer, the hollow fiber membrane module satisfying the following items:
(a) the water content of the hollow fiber membrane is 10% by weight or less relative to the tare weight of the hollow fiber membrane,
(b) the hydrophobic polymer contains no nitrogen, the hydrophilic group-containing polymer contains nitrogen, and the nitrogen content of the hollow fiber membrane is 0.05% by weight or more and 0.4% by weight or less,
(c) the content of the hydrophilic group-containing polymer in the inner surface of the membrane is 20% by weight or more and 45% by weight or less, and
(d) the consumption amount of an aqueous potassium permanganate solution (2.0×10⁻³ mol/L) used for titrating an eluted substance in 10 mL of a last part of a priming liquid is 0.2 mL or less per 1 m² of a membrane area.
[2]

The hollow fiber membrane module according to [1], wherein the number of deposited human platelets in the inner surface of the hollow fiber membrane is 20 platelets/(4.3×10³ μm²) or less.
[3]

The hollow fiber membrane module according to [1] or [2], wherein the hydrophilic group-containing polymer has a pyrrolidone group.
[4]

The hollow fiber membrane module according to any one of [1] to [3], wherein the hydrophilic group-containing polymer has an ester group.
[5]

The hollow fiber membrane module according to [4], wherein the ester group is derived from at least one selected from a vinyl carboxylic acid ester, an acrylic acid ester, and a methacrylic acid ester.
[6]

The hollow fiber membrane module according to any one of [3] to [5], wherein the hydrophilic group-containing polymer is a copolymer of vinyl acetate with vinylpyrrolidone.
[7]

The hollow fiber membrane module according to any one of [1] to [6], wherein the hydrophobic polymer is a polysulfone-based polymer.
[8]

A method for producing a hollow fiber membrane used in the hollow fiber membrane module according to any one of [1] to [7], the method including a step of using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.
[9]

A method for producing a hollow fiber membrane, the method including using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.
[10]

The method for producing a hollow fiber membrane according to [8] or [9], wherein the hydrophilic group of the hydrophilic group-containing polymer includes a pyrrolidone group.
[11]

The method for producing a hollow fiber membrane according to any one of [8] to [10], wherein the hydrophilic group-containing polymer has an ester group.
[12]

The method for producing a hollow fiber membrane according to [11], wherein the ester group is derived from at least one selected from a vinyl carboxylic acid ester, an acrylic acid ester, and a methacrylic acid ester.
[13]

The method for producing a hollow fiber membrane according to any one of [10] to [12], wherein the hydrophilic group-containing polymer is a copolymer of vinyl acetate with vinylpyrrolidone.
[14]

The method for producing a hollow fiber membrane according to any one of [8] to [13], wherein the hydrophobic polymer is a polysulfone-based polymer.
[15]

A method for producing a hollow fiber membrane module, the method including building the hollow fiber membrane produced by the method according to any one of [8] to [14] in a case.
[16]

The method for producing a hollow fiber membrane module according to [15], wherein irradiation with radiation is performed in a state where the water content of the hollow fiber membrane is adjusted to 10% by weight or less relative to the tare weight of the hollow fiber membrane built in the module.

### Advantageous Effects of Invention

According to the present invention, it is possible to obtain a dry-type hollow fiber membrane module with little eluted substance in which a hollow fiber membrane is simply hydrophilized, thereby improving blood compatibility and also suppressing elution of a hydrophilic group-containing polymer.

### BRIEF DESCRIPTION OF THE DRAWINGS

FIG. 1 is a schematic view (side view) showing one aspect of a hollow fiber membrane module of the present invention.

### DESCRIPTION OF EMBODIMENTS

The hollow fiber membrane module of the present invention is a hollow fiber membrane module including a built-in hollow fiber membrane including a hydrophobic polymer and a hydrophilic group-containing polymer.

### [Hollow Fiber Membrane Module]

The hollow fiber membrane module of the present invention can be used to separate into an objective substance to be recovered, and a waste, but is preferably used in applications including a blood purifier in which a liquid to be treated is allowed to flow to the inside of a hollow fiber membrane since an inner surface of a hollow fiber membrane made of a hydrophobic polymer is hydrophilized by a hydrophilic group-containing polymer. Examples of the blood purifier include a dialyzer and a hemofilter which are generally called an artificial kidney; a slow-type hemofilter and a hemodialysis filter for critical care; and the like.

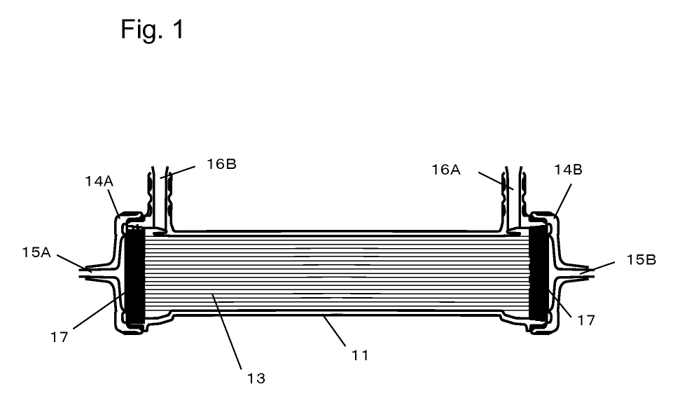

FIG. 1 is a schematic view showing one aspect of a hollow fiber membrane module of the present invention. The hollow fiber membrane module of the present invention preferably includes a case and a hollow fiber membrane module. A bundle of hollow fiber membranes 13 cut into a required length is preferably housed in a cylindrical case 11. Both ends of the hollow fiber membrane are preferably fixed to both ends of the cylindrical case by a potting material, or the like. At this time, both ends of the hollow fiber membrane are preferably opened.

The hollow fiber membrane module of the present invention preferably includes headers 14A and 14B at both ends of the case. The header 14A preferably includes an inlet 15A of the liquid to be treated. The header 14B preferably includes an outlet 15B of the liquid to be treated.

As shown in FIG. 1, the hollow fiber membrane module of the present invention preferably includes nozzles 16A and 16B at the side of the case in the vicinity of both ends of the case.

Usually, a liquid to be treated is introduced through the inlet 15A of the liquid to be treated, passed through the inside of the hollow fiber membrane, and then discharged through the outlet 15B of the liquid to be treated. Meanwhile, a process liquid is usually introduced through the nozzle 16A (the inlet of the process liquid), passed through the outside of the hollow fiber membrane, and then discharged through the nozzle 163 (the outlet of the process liquid). In other words, a flow direction of the liquid to be treated and a flow direction of the process liquid are usually opposed to each other.

There is no particular limitation on applications of the hollow fiber membrane module of the present invention. When used for artificial kidney application (blood purification application), blood as a liquid to be treated is usually introduced through the inlet 15A of the liquid to be treated and artificially dialyzed by passing through the inside of the hollow fiber membrane, and then blood after purification as an objective substance to be recovered is discharged through the outlet 15B of the liquid to be treated. In other words, a passage from the inlet 15A of the liquid to be treated to the outlet 15B of the liquid to be treated through the inside of the hollow fiber membrane becomes a passage (blood side passage) of the liquid to be treated. Hereinafter, this passage is sometimes referred to simply as a “blood side passage”.

Meanwhile, a dialyzate solution used as a process liquid is introduced through a nozzle 16A (the inlet of the process liquid) and the liquid to be treated (blood) is purified (dialyzed) by passing through the outside of the hollow fiber membrane, and then the dialyzate solution containing a toxic component (waste) in blood is discharged through the nozzle 16B (the outlet of the process liquid). In other words, a passage from the nozzle 16A to the nozzle 16B through the outside of the hollow fiber membrane becomes a passage (dialyzate solution passage) of the process liquid. Hereinafter, this passage is sometimes referred to simply as a “dialyzate solution passage”.

### [Hydrophobic Polymer and Hydrophilic Group-Containing Polymer]

The hydrophobic polymer in the present invention refers to a polymer, which is slightly soluble or insoluble in water, solubility in 100 g of pure water at 20° C. being less than 1 g. Meanwhile, the hydrophilic group-containing polymer refers to a polymer having a hydrophilic group, solubility in 100 g of pure water at 20° C. of a polymer having a hydrophilic group alone being 10 g or more. In the present invention, the hydrophilic group refers to a minimum unit capable of polymerizing alone, and examples of such hydrophilic group include acrylamide, acrylic acid, N-vinyl-2-pyrrolidone, vinyl alcohol, and the like.

It is important that the hollow fiber membrane module of the present invention satisfies the following items:
(a) the water content of the hollow fiber membrane is 10% by weight or less relative to the tare weight of the hollow fiber membrane,
(b) the hydrophobic polymer contains no nitrogen, the hydrophilic group-containing polymer contains nitrogen, and the nitrogen content of the hollow fiber membrane is 0.05% by weight or more and 0.4% by weight or less,
(c) the content of the hydrophilic group-containing polymer in the inner surface of the membrane is 20% by weight or more and 45% by weight or less, and
(d) the consumption amount of an aqueous potassium permanganate solution (2.0×10⁻³ mol/L) used for titrating an eluted substance in 10 mL of a last part of a priming liquid is 0.2 mL or less per 1 m² of a membrane area:

### [Hollow Fiber Membrane and Water Content Thereof]

Too large water content of the hollow fiber membrane module may cause a fear of bacterial growth during storage or may cause freezing of the hollow fiber membrane, leading to deterioration of performance. Meanwhile, a low water content dry-type one enables weight saving of the hollow fiber membrane module, which leads to reduced transport cost and an improved safety. In a hollow fiber membrane module with a substantially dry hollow fiber membrane, defoamability during use is improved. Thus, the water content in the hollow fiber membrane of the hollow fiber membrane module according to the present invention is adjusted to 10% by weight or less, preferably 4% by weight or less, andmore preferably 2% by weight or less, relative to the tare weight of the hollow fiber membrane. There is no particular limitation on the lower limit, and the lower limit is substantially 0%.

Here, the water content in the present invention is calculated by the equation: water content (% by weight)=100×(a−b)/b, where the symbol (a) denotes the mass of a hollow fiber membrane module or a hollow fiber bundle before drying, and the symbol (b) denotes the mass of a hollow fiber membrane module or a hollow fiber bundle after drying the hollow fiber membrane until reaching an absolute dry condition.

The hollow fiber membrane built in the hollow fiber membrane module is preferably a membrane having an asymmetric structure composed of a layer contributing to the separation performance and a supporting layer contributing to the mechanical strength of the membrane in view of permeability and separation performance. Particularly in a dialysis membrane in which blood is allowed to pass through the inside of a hollow fiber, hydrophilicity of the inner surface of the hollow fiber in view of blood compatibility. Therefore, blood compatibility is improved by enhancing hydrophilicity of the inner surface of the hollow fiber.

### [Hydrophobic Polymer Containing No Nitrogen]

The hydrophobic polymer serving as a membrane material contains no nitrogen and examples thereof include, but are not limited to, polysulfone-based polymer, polystyrene, polyethylene, polypropylene, polycarbonate, polyvinylidene fluoride, and the like.

In the present invention, the phrase “hydrophobic polymer contains no nitrogen” means that the hydrophobic polymer substantially contains no nitrogen atom. The content of nitrogen obtained based on trace nitrogen analysis is 500 ppm or less, preferably 300 ppm or less, more preferably 100 ppm or less, and particularly preferably detection limit or less. Most preferably, the hydrophobic polymer contains no nitrogen.

The polysulfone-based polymer is suited to form a hollow fiber membrane, and is suitably used since it has strong interactions with an ester group of vinyl acetate and also makes it easy to introduce a hydrophilic group-containing polymer having the ester group as the hydrophobic group into the hollow fiber membrane. The polysulfone-based polymer has an aromatic ring, a sulfonyl group, and an ether group in the main chain, and examples thereof include polysulfone, polyether sulfone, polyallylether sulfone, and the like. For example, polysulfone-based polymers represented by the below-mentioned chemical formulas (1) and (2) are suitably used. Of these polysulfone-based polymers, polysulfone (below-mentioned formula (1)) is particularly preferably used, but the polysulfone-based polymer is not limited thereto in the present invention. In the formulas, n is an integer of, for example, 50 to 80.

### Formulas (1) and (2)

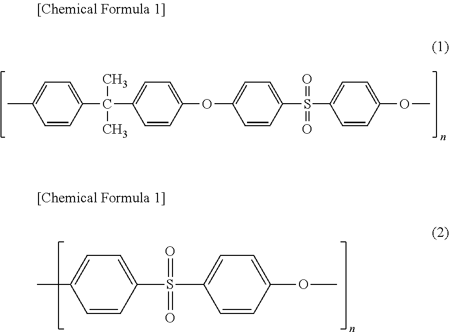

Specific examples of the polysulfone include polysulfones such as Udel polysulfone P-1700, P-3500 (manufactured by Solvay S.A.), Ultrason 53010, S6010 (manufactured by BASF Corporation), VICTREX (manufactured by Sumitomo Chemical Company, Limited), Radel A (manufactured by Solvay S.A.), and Ultrason E (manufactured by BASF Corporation). The polysulfone-based polymer used in the present invention is preferably a polymer composed only of repeating units represented by the formulas (1) and/or (2), and other monomers may be copolymerized as long as the effects of the present invention are not impaired. The copolymerization ratio of the other copolymerized monomer is preferably 10% by weight or less, although there is no particular limitation.

### [Hydrophilic Group-Containing Polymer Containing Nitrogen]

The hydrophilic group-containing polymer used in the present invention may be those containing nitrogen. Examples of the hydrophilic group-containing polymer containing nitrogen include polyethyleneimine, polyvinylpyrrolidone, and the like. Of these, a polymer having a pyrrolidone group is preferable from the viewpoint of improving blood compatibility.

From the viewpoint of safety and economy, polyvinylpyrrolidone is particularly preferable.

It is also possible to use, as the hydrophilic group-containing polymer, a hydrophilic group-containing polymer having a hydrophobic group, and use of the polymer having a hydrophobic group is effective since affinity with the hydrophobic polymer as the membrane material is improved and the hydrophobic interaction enables the introduction of the hydrophilic group-containing polymer, more efficiently. The hydrophobic group as used herein is defined as a repeating unit which is slightly soluble or insoluble in water in the case of a polymer thereof alone, and the phrase “slightly soluble or insoluble in water” means that the solubility in 100 g of pure water at 20° C. is less than 1 g. It is preferred that the hydrophobic group has an ester group from the viewpoint of blood compatibility, although its mechanism is not interpreted in detail.

Accordingly, in the present invention, it is preferred that the hydrophilic group-containing polymer has an ester group.

Specific examples of such hydrophobic group (ester group) include, but are not limited to, vinyl carboxylic acid esters such as vinyl acetate; acrylic acid esters such as methyl acrylate and methoxyethyl acrylate; methacrylic acid esters such as methyl methacrylate, ethyl methacrylate, and hydroxyethyl methacrylate; and the like. It is preferred to have an ester group derived therefrom.

In other words, in the present invention, it is more preferred that the hydrophilic group-containing polymer has an ester group and also the ester group is derived from at least one selected from a, vinyl carboxylic acid ester, an acrylic acid ester, and a methacrylic acid ester.

In the present invention, it is particularly preferred that a copolymer composed of vinyl acetate and vinylpyrrolidone is used as the hydrophilic group-containing polymer, from the viewpoint of efficiency of production into a membrane material and blood compatibility.

Meanwhile, small proportion of the hydrophobic group in the hydrophilic group-containing polymer weakens the interaction with the hydrophobic polymer as the membrane material, and thus the hydrophilic group-containing polymer having a hydrophobic group is less likely to obtain a merit of improving introduction efficiency. Meanwhile, large proportion of the hydrophobic group may cause deterioration of hydrophilicity of the inner surface of the hollow fiber membrane, leading to deterioration of blood compatibility. Therefore, the proportion of the hydrophobic group is preferably 20 mol % or more, and more preferably 30 mol % or more, while the proportion is preferably 80 mol % or less, and more preferably 70 mol % or less.

In the present invention, in order to obtain the objective applications and properties, not only the hydrophilic group-containing polymer is used alone, but also different types of hydrophilic group-containing polymers may be appropriately used in combination.

As long as the effects of the present invention are not impaired, a polymer containing no nitrogen may be used without any problem. Specific examples thereof include, but are not limited to, polyethylene glycol, polyvinyl alcohol, carboxymethyl cellulose, polypropylene glycol, and the like.

### [Nitrogen Content of Hollow Fiber Membrane]

In the present invention, since no nitrogen atom is contained in the hydrophobic polymer, the nitrogen atom contained in the hollow fiber membrane is mainly derived from a hydrophilic group-containing polymer which is used mainly for the purpose of imparting hydrophilicity or controlling the structure, and it can be said that the hydrophilic group-containing polymer is a compound capable of causing elution, including the case where a hydrophilic group-containing polymer containing a nitrogen atom, and other low molecular weight compounds exist. Particularly in the hollow fiber membrane in which a hydrophobic polymer is composed of a polysulfone-based polymer, PVP is often used as the hydrophilic group-containing polymer from the viewpoint of compatibility. Since a nitrogen atom is contained in a pyrrolidone group, the measurement of the nitrogen content enables determination of an index of the content of components including the content of the hydrophilic group-containing polymer included in the entire hollow fiber membrane. Large content of the hydrophilic group-containing polymer included in the hollow fiber membrane may lead to an improvement in permeability since the entire membrane is hydrophilized. Meanwhile, too large content may cause a problem such as an increase in eluted substance. Therefore, the nitrogen content of the hollow fiber membrane is preferably 0.05% by weight or more, more preferably 0.1% by weight or more, and still more preferably 0.15% by weight or more. The upper limit is preferably 0.4% by weight or less, More preferably 0.38% by weight or less, and still more preferably 0.35% by weight or less.

The nitrogen content in the present invention can be measured from oxidative decomposition using trace nitrogen analysis by a reduced-pressure chemiluminescence method. An example of detailed conditions is shown in Examples. An average of the results obtained by measuring three times is used as a measured value.

### [Content of Hydrophilic Group-Containing Polymer in Inner Surface of Hollow Fiber Membrane]

In the present invention, it is desired that the hydrophilic group-containing polymer is localized inside the hollow fiber membrane, which usually become a surface in contact with the liquid to be treated, in blood purification application. The content of the hydrophilic group-containing polymer in the inner surface of the hollow fiber membrane is 20% by weight or more, preferably 22% by weight or more, and more preferably 25% by weight or more. If the content of the hydrophilic group-containing polymer is less than 20% by weight, blood compatibility deteriorates because of poor hydrophilicity, so that blood coagulation is likely to occur. Meanwhile, if the content of the hydrophilic group-containing polymer exceeds 45% by weight, the content of the hydrophilic group-containing polymer eluted in blood may increase, thus causing side effects during long-term dialysis and complication due to the eluted polymer. If the nitrogen content of the entire hollow fiber membrane and the content of the hydrophilic group-containing polymer of the inner surface are too large, irradiation with radiation may cause excess proceeding of crosslinking of polymers, leading to deterioration of biocompatibility. Therefore, the content of the hydrophilic group-containing polymer is 45% by weight or less, and preferably 42% by weight or less.

In the present invention, the content of the hydrophilic group-containing polymer in the inner surface of the hollow fiber membrane can be measured using X-ray photoelectron spectroscopy (XPS). Values measured at an angle of 90° is used as a measurement angle. At a measurement angle of 90°, a region from the surface to a depth of about 10 nm can be detected. The average of values measured at three places should be used. For example, when the hydrophobic polymer is polysulfone and the hydrophilic group-containing polymer is polyvinylpyrrolidone, the content (% by weight) of vinylpyrrolidone of the inner surface of the hollow fiber membrane can be calculated from the nitrogen content (c (atomic %)) and the sulfur content (d (atomic %)) according to the following equation: polyvinylpyrrolidone content (f)=100×(c×111)/(c×111+d×442), where 111 is a molecular weight of a vinylpyrrolidone group, and 442 is a molecular weight of a repeating unit constituting polysulfone.

When using a hydrophilic group-containing polymer having an ester group, the content of an ester group existing in the inner surface of the hollow fiber membrane is taken into consideration from the viewpoint of blood compatibility. High ester group content of the inner surface may cause strong hydrophobicity, leading to deterioration of blood compatibility and deterioration of separation performance. Therefore, the content of carbon derived from an ester group of the inner surface is preferably 10 atomic % or less, and more preferably 5 atomic % or less.

The content of carbon derived from an ester group existing in the inner surface of the hollow fiber membrane can be measured using X-ray photoelectron spectroscopy (XPS). Values measured at an angle of 90° are used. At a measurement angle of 90°, a region from the surface to a depth of about 10 nm is detected. The average of values measured at three places are used. The carbon peak, derived from an ester group (COO) can be determined by deconvoluting peaks observed in the range from the main C1s peak derived from CH or C—C to the peak at +4.0 to +4.2 eV. The content of carbon derived from an ester group (atomic %) is determined by calculating the ratio of the corresponding peak area to the peak area for all elements. More specifically, C1s peaks are composed of five components: a component mainly derived from CHx, C—C, C═C, C—S; a component mainly derived from C—O, C—N; a component derived from π-π* satellite; a component derived from C═O; and a component derived from COO. Therefore, the peaks are deconvoluted into the five components. The COO-derived component corresponds to the peak observed at +4.0 to +4.2 eV from the main CHx or C—C peak (at about 285 eV). When calculated, the first decimal place of the peak area ratio of each component is rounded off. The ester carbon content may be calculated by multiplying the C1s carbon content (atomic %) by the peak area ratio of the COO-derived component. As a result of peak deconvolution, a ratio of 0.4% or less is determined to be the detection limit.

It is also possible to determine the content (% by weight) of vinyl acetate of the surface of the hollow fiber membrane utilizing the above method. For example, the hydrophilic group-containing polymer having an ester group is a copolymer of vinylpyrrolidone with vinyl acetate in a molar ratio of 6/4, the vinyl acetate, content of the surface of the hollow fiber membrane can be calculated from the nitrogen content (c (atomic %)), the sulfur content (d (atomic %)), and the content of carbon derived from an ester group (e (atomic %)) according to the following equation: the content (g (% by weight)) of vinyl acetate of the surface of the hollow fiber membrane=(e×86/(c×111+d×442+e×86))×100, since a molecular weight of a vinylpyrrolidone group is 111, a molecular weight of a repeating unit constituting polysulfone is 442, and a molecular weight of vinyl acetate is 86.

Therefore, when the hydrophilic group-containing polymer is a copolymer of vinylpyrrolidone with vinyl acetate, the hydrophilic group-containing polymer content of the inner surface of the hollow fiber membrane can be represented by the sum of the vinylpyrrolidone content (f) and the vinyl acetate content (g).

Content of hydrophilic group-containing polymer (h (% by weight)) of inner surface of hollow fiber membrane=f+g.

### [Content of Hydrophilic Group-Containing Polymer in Outer Surface of Hollow Fiber Membrane]

It is also possible to measure the content of the hydrophilic group-containing polymer of the outer surface of the hollow fiber membrane using XPS in the same way as the inner surface. When the content of the hydrophilic group-containing polymer of the outer surface is high, there sometimes arise problems such as fixation of hollow fiber membranes via a hydrophilic group-containing polymer during drying, and deterioration of assemblability of a module. From the viewpoint of preventing penetration of endotoxin contained in a dialyzate solution, it becomes more effective as the content of the hydrophilic group-containing polymer of the outer surface more decreases. In the case of a dry fiber, small hydrophilic group-containing polymer content of the outer surface may cause deterioration of priming properties since it is not easy to be wetted.

Thus, the content of the hydrophilic group-containing polymer of the outer surface is preferably 45% by weight or less, and more preferably 40% by weight or less, while the lower limit is preferably 20 mass % or more.

### [State of Hydrophilic Group-Containing Polymer Existing in Inner Surface of Hollow Fiber Membrane]

It is desired that the hydrophilic group-containing polymer uniformly exists in the inner surface of the hollow fiber membrane in view of blood compatibility. Distribution of the hydrophilic group-containing polymer can be measured by total reflection infrared spectroscopy (ATR). ATR measuring method is as follows: infrared absorption spectrum is measured at 25 points in a measurement area of 3 μm×3 μm with a cumulative number of 30 or more. The 25-point measurement is performed at three different places per one hollow fiber membrane, with respect to three hollow fiber membranes per one module. A base line is drawn on the resulting infrared absorption spectrum in the range of 1,620 to 1,711 cm⁻¹, and the peak area surrounded by the base line and the positive part of the spectrum is determined to be the peak area (A_{NCO}) derived from polyvinylpyrrolidone. In other words, (A_{NCO}) is defined as the area of the positive region of the spectrum in the wavelength range of 1,620 to 1,711 cm⁻¹ . Similarly, a base line is drawn on the spectrum in the range of 1,549 to 1,620 cm⁻¹, and the peak area surrounded by the base line and the positive part of the spectrum is determined to be the peak area (A_{CC}) derived from the benzene ring C═C of polysulfone. The ratio between them (A_{CO})/(A_{CC}) is then calculated. The average of (A_{NCO})/(A_{CC}) is preferably 0.4 or more, more preferably 0.6 or more, and still more preferably 0.7 or more. The proportion of the measurement points, at which the ratio (A_{NCO})/(A_{CC}) is 0.25 or less, is preferably 10% or less, and more preferably 5% or less, based on the total measurement points (25 points).

When the hydrophilic group-containing polymer has an ester group, distribution of the ester group can be measured by ATR measurement, similarly. A base line is drawn on the resulting infrared absorption spectrum in the range of 1,711 to 1,750 cm⁻¹, and the peak area surrounded by the base line and the positive part of the spectrum is determined to be the peak area (A_{COO}) derived from an ester group, and then a ratio of the peak area (_{ACOO}) to the peak area (A_{CC}) derived from the benzene ring C═C of polysulfone (A_{COO})/(A_{CC}) is calculated. An average of the ratio (A_{COO})/(A_{CC}) is preferably 0.005 or more, more preferably 0.01 or more, and still more preferably 0.02 or more. The proportion of the measurement points, at which the ratio (A_{COO})/(A_{CC}) is 0.001 or less, is preferably 10% or less, and more preferably 5% or less, based on the total measurement points (25 points).

### [Consumption Amount of Aqueous Potassium Permanganate Solution to Last Part of Priming Liquid]

An index to obtain high safety includes the consumption amount in the case of potassium permanganate titration of an eluted substance which is eluted in a liquid when allowed to pass through a passage of a membrane.

In the present invention, a last part of a priming liquid is selected as the above liquid. Here, the last part of a priming liquid is a liquid obtained by allowing ultrapure water heated to 37° C. to pass through a passage (blood side passage) at the side of the liquid to be treated of a hollow fiber membrane module at a rate of 100 mL/min for 7 minutes, allowing the liquid to pass through a passage (dialyzate side passage) at the process liquid side at a rate of 500 mL/min for 5 minutes, and sampling 200 mL of the liquid which flows out during last 2 minutes in the case of allowing the liquid to pass through a passage (blood side passage) at the side of the liquid to be treated at a rate of 100 mL/min for 3 minutes, again.

After collecting 10 mL of a sampling liquid from the obtained sampling liquid, the sampling liquid thus collected is subjected to a test. To 10 mL of a last part of a priming liquid, 20 mL of an aqueous potassium permanganate solution (2.0×10⁻³ mol/L) and 1 mL of 101 by volume of sulfuric acid, and a boiling stone were added, followed by boiling for 3 minutes. Then, the mixture was cooled to room temperature (20 to 30° C.) (preferably cooled by allowing to cool for 10 minutes). Thereafter, the mixture is well cooled with iced water (preferably cooled for 10 minutes). After adding 1 mL of an aqueous 10% by weight potassium iodide solution, the mixture was well stirred in a state at 20° C. to 30° C. and allowed to stand for 10 minutes, followed by titration with an aqueous sodium thiosulfate solution (1.0×10⁻² mol/L). At the time when color of the solution turns pale yellow, 0.5 mL of an aqueous 1% by weight starch solution was added, followed by well, stirring at 20° C. to 30° C. Thereafter, titration is performed until color of the solution turns transparent.

A difference between the amount of the aqueous sodium thiosulfate solution required for titration of ultrapure water which was not allowed to pass through the hollow fiber membrane module, and the amount of the aqueous sodium thiosulfate solution required for titration of the last part of a priming liquid is defined as the amount of the aqueous potassium permanganate solution consumed by the eluted substance (consumption amount of the aqueous potassium permanganate solution).

If numerous eluted substance is eluted from the hollow fiber membrane, the eluted substance is mixed into blood during long-term dialysis, so that side effects and complication may occur. Therefore, the consumption amount of the aqueous potassium permanganate solution is preferably 0.2 mL or less, more preferably 0.15 mL or less, still more preferably 0.1 mL or less, and most preferably 0 mL, per 1 m² of the membrane area.

### [Number of Platelets Deposited to Inner Surface of Hollow Fiber Membrane]

Blood compatibility in the inner surface of the hollow fiber membrane can be evaluated by the number of platelets deposited to the hollow fiber membrane. Since a large number of deposited platelets may lead to blood coagulation, it can be said that the inner surface of the hollow fiber membrane has poor blood compatibility. The number of platelets deposited to the inner surface of the hollow fiber membrane can be evaluated by observing the inner surface of the hollow fiber membrane after being in contact with human blood using a scanning electron microscope. When the inner surface of the sample is observed at a magnification of 1,500 times, the number of the deposited platelets per field (4.3×10³ μm²) is preferably 20 platelets or less, more preferably 10 platelets or less, still more preferably 8 platelets or less, and particularly preferably 4 platelets or less. An average (obtained by rounding off the second decimal position) of the number of the deposited platelets observed different ten fields is used.

### [Method for Producing Hollow Fiber Membrane and Hollow Fiber Membrane Module]

Subsequently, a method for producing a hollow fiber membrane and a hollow fiber membrane module will be described.

In the present invention, a hollow fiber membrane is preferably produced by using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.

More specifically, a method for producing a hollow fiber membrane of the present invention preferably includes a step of discharging a membrane forming stock solution and an injection liquid through a double annulation spinneret, wherein a solution which contains a hydrophobic polymer containing no nitrogen is used as a membrane forming stock solution, and a solution which contains 0.01% by weight or more and 1% by weight or less of a hydrophilic group-containing polymer containing nitrogen is used as an injection liquid.

More preferably, in the step, a membrane forming stock solution is discharged through a slit part of a double annulation spinneret, and an injection liquid is discharged through a circular tube part.

In the step, the membrane forming stock solution preferably contains a hydrophobic polymer, and a good solvent thereof and a poor solvent thereof.

The method for producing a hollow fiber membrane of the present invention preferably includes, after the step of discharging a membrane forming stock solution and an injection liquid through a double annulation spinneret, a step of introducing the discharged substance into the dry part (allowing to pass the discharged substance through the dry part), and coagulating the discharged substance in a coagulation bath to obtain a hollow fiber membrane.

In other words, in the present invention, a hollow fiber membrane is preferably produced by discharging a membrane forming stock solution containing a hydrophobic polymer, a good solvent thereof, and a poor solvent thereof through a slit part of a double annulation spinneret, discharging the injection liquid through a circular tube part, allowing to pass through a dry part, and coagulating in a coagulation bath.

The mechanical strength of the hollow fiber membrane can be increased by increasing the concentration of the hydrophobic polymer in the membrane forming stock solution. Meanwhile, too large concentration of the hydrophobic polymer may cause problems such as decrease in solubility and poor discharge due to an increase in viscosity of the membrane forming stock solution. The concentration of the hydrophobic polymer enables the adjustment of permeability and molecular weight cutoff. Increase in concentration of the hydrophobic polymer may cause an increase in density of the inner surface of hollow fiber membrane, leading to deterioration of permeability and molecular weight cutoff. Thus, the concentration of the hydrophobic polymer in the membrane forming stock solution is preferably 14% by weight or more, while the concentration of the hydrophobic polymer is preferably 24% by weight or less.

The good solvent in the present invention means a solvent which substantially dissolves a hydrophobic polymer in the membrane forming stock solution. When using a polysulfone-based polymer, N,N-dimethylacetamide is suitably used because of its solubility, although there is no particular limitation. Meanwhile, the poor solvent means a solvent which does not substantially dissolve a hydrophobic polymer at the membrane forming temperature. Water is suitably used, although there is no particular limitation.

The addition of the poor solvent to the membrane forming stock solution accelerates proceeding of phase separation since the poor solvent serves as a nucleus. Meanwhile, too large additive amount of the poor solvent makes the membrane forming stock solution unstable, and thus it becomes hard to obtain reproducibility in membrane formation. Optimum additive amount of the poor solvent varies depending on the type of poor solvent. When using water as typical poor solvent, the additive amount of the poor solvent in the membrane forming stock solution is preferably 0.5% by weight or more, while the additive amount of the poor solvent is preferably 4% by weight or less.

There have hitherto been used, as a method for introducing a hydrophilic group-containing polymer into the inner surface of the hollow fiber membrane, a method in which a hydrophilic group-containing polymer is mixed in a membrane forming stock solution of a hollow fiber membrane, followed by forming, a method in which a hydrophilic group-containing polymer is added to an injection liquid during membrane formation, and a method in which a surface of a membrane is coated with a hydrophilic group-containing polymer after forming a hollow fiber membrane.

In the present invention, it is preferred to use a method in which a hydrophilic group-containing polymer is added to an injection liquid during membrane formation and then a hydrophilic group-containing polymer is introduced into an inner surface of a hollow fiber membrane by discharging together with a stock solution. Use of the method enables dense coating of the surface of the hollow fiber membrane with the hydrophilic group-containing polymer even if a small amount of the hydrophilic group-containing polymer is used, thus making it possible to suppress an eluted substance. Because of being coated with the hydrophilic group-containing polymer during membrane formation, drying can be performed in a spinning step and there is no need to use a special facility, and also a hollow fiber membrane module having blood compatibility can be obtained. Therefore, the method is a suitable method in the present invention.

A method of coating a surface of a membrane with a hydrophilic group-containing polymer after forming a hollow fiber membrane is also a suitable method. As mentioned below, this method also enables dense coating of the surface of the hollow fiber membrane with the hydrophilic group-containing polymer by elaborating conditions such as concentration and temperature of a solution used for coating, and a flowing method of a coating liquid, thus making it possible to suppress an eluted substance.

Even when using, as a method for introducing a hydrophilic group-containing polymer into an inner surface of a hollow fiber membrane, either a method of adding to an injection liquid during membrane forming or a method in which a surface of a membrane is coated after forming a hollow fiber membrane, it is possible to expect an improvement in permeability and a further improvement in hydrophilicity due to the effect of a pore forming material by separately adding a hydrophilic group-containing polymer to a membrane forming stock solution. Too large additive amount of the hydrophilic group-containing polymer in the membrane forming stock solution may cause a decrease in solubility and poor discharge due to an increase in viscosity of the membrane forming stock solution, and also remaining of a large amount of the hydrophilic group-containing polymer in the hollow fiber membrane may cause deterioration of permeability due to an increase in permeation resistance. Optimum amount of the hydrophilic group-containing polymer to be added to the membrane forming stock solution varies depending on the type and objective performance, and is preferably 1% by weight or more, while the optimum amount is preferably 15% by weight or less. There is no particular limitation on the hydrophilic group-containing polymer to be added to the membrane forming stock solution and, when a polysulfone-based polymer is used as the hydrophobic polymer, polyvinylpyrrolidone is suitably used because of its high compatibility.

The polymer is preferably melted at high temperature so as to improve solubility, but may cause denaturation of the polymer due to heat, and change in composition due to vaporization of the solvent. Therefore, the melting temperature is preferably 30° C. or higher and 120° C. or lower. Optimum range of the melting temperature sometimes varies depending on the type of the hydrophobic polymer and additives.

The injection liquid used during formation of a hollow fiber membrane is a mixed solution of a good solvent and a poor solvent, and permeability and molecular weight cutoff of the hollow fiber membrane can be adjusted by the ratio between them. There is no particular limitation on poor solvent, and water is suitably used. There is no particular limitation on good solvent, and N,N-dimethylacetamide is suitably used.

When the membrane forming stock solution is in contact with the injection liquid, phase separation of the membrane forming stock solution is induced by the action of the poor solvent and thus coagulation proceeds. When the ratio of the poor solvent in the injection liquid is excessively increased, permeability and molecular weight cutoff of the membrane deteriorate. Meanwhile, when the ratio of the poor solvent in the injection liquid is excessively increased, the solution is dropped in a state of liquid, thus failing to obtain a hollow fiber membrane. Proper ratio of both solvents in the injection liquid varies depending on the type of the good solvent and the poor solvent. The proportion of poor solvent is preferably 10% by weight or more in the mixed solvent of both solvents, while the proportion is preferably 80% by weight or less.

When the hydrophilic group-containing polymer is added to the injection liquid, numerous hydrophilic group-containing polymer can be selectively introduced into the inner surface of the hollow fiber membrane. This is because the hydrophilic group-containing polymer is also incorporated into the inner surface by causing diffusion of the hydrophilic group-containing polymer in the injection liquid in the stock solution when the injection liquid is diffused in the stock solution, thereby inducing phase separation. Therefore, entanglement between the hydrophilic group-containing polymer and molecules of the membrane material arises, so that it is possible to firmly bond to the membrane material as compared with the case where the hydrophilic group-containing polymer is imparted after membrane formation, thus making it possible to reduce an eluted substance. In this way, since the hydrophilic group-containing polymer is introduced into the inner surface by diffusion of the hydrophilic group-containing polymer during membrane formation, the length of the dry part after discharging the stock solution, that is, the dry part length becomes important as spinning conditions. When the dry part length is too short, diffusion of the hydrophilic group-containing polymer may not proceed, thus failing to sufficiently coat the inner surface. Therefore, the dry part length is preferably 50 mm or more, and more preferably 100 mm or more. Meanwhile, when the dry part length is too long, diffusion may proceed and thus the hydrophilic group-containing polymer reaches the outer surface, and spinning stability may deteriorate by fiber sway. Therefore, the dry part length is preferably 600 mm or less. A large influence is exerted by the concentration of the good solvent in the injection liquid. It is considered that low concentration of good solvent excessively accelerates coagulation of the inner surface and thus diffusion of the hydrophilic group-containing polymer is less likely to proceed, while high concentration of good solvent suppresses coagulation of the inner surface, leading to excess proceeding of diffusion of the hydrophilic group-containing polymer. Therefore, in the injection liquid, the concentration of the good solvent in both solvents is preferably 40% by weight or more, and more preferably 50% by weight or more, while the concentration of good solvent is preferably 90% by weight or less, more preferably 80% by weight or less, and still more preferably 70% or less.

Here, it has been considered that a sufficient amount of the hydrophilic group cannot be imparted if the amount of the hydrophilic group-containing polymer to be added to the injection liquid is about 10% by weight in the injection liquid. However, the addition of such large amount of the hydrophilic group may cause an increase in an eluted substance. It has been found that, in the production of a dry-type hollow fiber membrane according to the present invention, design of the injection liquid containing the hydrophilic group-containing polymer can sufficiently impart hydrophilicity to the hollow fiber membrane by the addition in a small amount. Meanwhile, too small amount of the hydrophilic group-containing polymer may cause insufficient hydrophilization of the inner surface of hollow fiber membrane, leading to deterioration of blood compatibility.

Therefore, in the present invention, the content of the hydrophilic group-containing polymer in the injection liquid is preferably 0.01% by weight or more, and more preferably 0.03% by weight or more, while the upper limit is preferably 1% by weight or less, more preferably 0. 5% by weight or less, and most preferably 0.1% by weight or less.

The temperature of a double annulation spinneret during discharging exerts an influence on viscosity of the membrane forming stock solution, phase separation behavior, and rate of diffusion of the injection liquid into the membrane forming stock solution. In general, the higher the temperature of the double annulation spinneret, permeability and molecular weight cutoff of the resulting hollow fiber membrane increase. Too high temperature of the double annulation spinneret may cause unstable discharging due to a decrease in viscosity of the membrane forming stock solution and deterioration of coagulant property, leading to deterioration of spinnability. Meanwhile, low temperature of the double annulation spinneret may cause deposition of water to the double annulation spinneret due to dew condensation. Therefore, the temperature of the double annulation spinneret is preferably 20° C. or higher, while the temperature of the double annulation spinneret. is preferably 90° C. or lower.

When the discharged membrane forming stock solution and the injection liquid pass through the dry part, diffusion of poor solvent in the injection liquid to the membrane forming stock solution proceeds to form a membrane structure in which the pore size increases from the inner surface of the hollow fiber side to the outer surface side. Furthermore, as mentioned above, when the injection liquid diffuses into the stock solution to cause phase separation, the hydrophilic group-containing polymer contained in the injection liquid is incorporated into the inner surface of the membrane.

At the dry part, when the outer surface is in contact with air, moisture in air is incorporated and serves as poor solvent, and thus phase separation proceeds. Therefore, open porosity of the outer surface can be adjusted by controlling a dew point of the dry part. If the dew point of the dry part is low, phase separation does not sometimes sufficiently proceed and open porosity of the outer surface may decrease, so that friction of the hollow fiber membrane increases, leading to deterioration of spinnability. Meanwhile, even when the dew point of the dry part is too high, the outer surface may be sometimes coagulated, leading to a decrease in open porosity. The dew point of the dry part is preferably 60° C. or lower, while the dew point is preferably 10° C. or higher.

A coagulation bath contains a poor solvent as a main component and a good solvent is optionally added. Water is suitably used as the poor solvent. When the membrane forming stock solution enters into the coagulation bath, the membrane forming stock solution is coagulated by a large amount of the poor solvent in the coagulation bath and the membrane structure is fixed. Since coagulation is suppressed by more increasing the temperature in the coagulation bath, permeability and molecular weight cutoff increase.

There is a need for the hollow fiber membrane obtained by coagulating in the coagulation bath to be washed with water since the hollow fiber membrane contains an excess hydrophilic group-containing polymer derived from the solvent and the stock solution.

Insufficient washing with water may lead to complicated washing before use, and also may cause a problem such as flow of the eluted substance into the liquid to be treated. Since an increase in water washing temperature leads to an increase in water washing efficiency, the temperature of water washing is preferably 50° C. or higher.

When the inner surface of the hollow fiber membrane is coated after forming a hollow fiber membrane, the concentration of the hydrophilic group-containing polymer of the coating liquid, the contact time, and the temperature during coating exert an influence on the amount of the hydrophilic group-containing polymer with which the inner surface of hollow fiber membrane is coated, and density. If the concentration of the hydrophilic group-containing polymer is too high, the hydrophilic group-containing polymer itself may be eluted, so that the concentration is preferably 0.08% by weight or less, and more preferably 0.05% by weight.or less. Meanwhile, if the concentration is too low, it is impossible to sufficiently coat the membrane surface with the hydrophilic group-containing polymer, leading to an increase in an eluted substance and deterioration of blood compatibility, so that the concentration is preferably 0.001% by weight or more, and more preferably 0.01% by weight or more.

Water is suitably used as the solvent used in the coating liquid in view of safety.

The temperature is suitably 20 to 80° C. and the contact time is suitably 10 seconds or more. It is possible to densely coat the membrane surface with the hydrophilic group-containing polymer by allowing the coating liquid to pass through in a membrane thickness direction.

Particularly, when using a hydrophilic group-containing polymer having a hydrophobic group, the temperature of the coating liquid exerts a large influence on or causes a change in affinity with a membrane material. In a polymer having a hydrophilic group and a hydrophobic group, the form of the interaction with the water molecule varies depending on the temperature of water, and the polymer is sometimes precipitated by forming a micelle in which hydrophobic groups are oriented on the surface. This temperature is called a clouding point. Although the details are not still clear, when using a hydrophilic group-containing polymer having a hydrophobic group on a hydrophobic surface, hydrophobic interaction between the membrane surface and the hydrophobic group in the hydrophilic group-containing polymer by coating at the temperature near the clouding point, thus making it possible to densely coat the membrane surface with the hydrophilic group-containing polymer in an efficient manner. For example, when using, as the hydrophilic group-containing polymer, vinylpyrrolidone/vinyl acetate (6/4 (molar ratio)) random copolymer (“KOLLIDON” (registered trademark) VA64″, manufactured by BASF Corporation), the clouding point is approximately about 70° C., so that the temperature of the coating liquid is suitably 60 to 80° C.

When coating is continuously performed, it is possible to more uniformly coat as a flow rate of a coating liquid more increases. If the flow rate is too large, the membrane surface may not be coated with a sufficient amount of the coating liquid, so that the flow rate is suitably within a range of 200 to 1,000 mL/min.

Examples of the method for producing a hollow fiber membrane module in which the water content of a hollow fiber membrane is 10% by weight or less include a method in which a hollow fiber membrane having the water content of 10% by weight or less obtained by drying before fabricating a module is formed into a bundle and then incorporated into a case to fabricate a module, and a method in which a hollow fiber membrane is dried after fabricating a hollow fiber membrane module. Although there is no particular limitation, when drying is performed after fabricating a module, the hollow fiber membrane is preferably dried before fabricating a module since there are problems that it takes a long time to adjust to the water content of 10% by weight or less by drying, and membranes may be fixed to each other in the case of drying a hollow fiber in a state of a bundle.

Examples of the method for subjecting a hollow fiber membrane to a drying treatment include a method in which drying is performed by hot air or microwave irradiation. Although there is no particular limitation, drying by hot air is suitably used in view of simplicity.

Drying by hot air may cause decomposition and deterioration of the hydrophilic group-containing polymer at high drying temperature, or may cause adhesion between hollow fiber membranes. Meanwhile, a drying treatment takes a long time at low drying temperature. Therefore, the drying temperature is preferably 50° C. or higher, and more preferably 70° C. or higher, while the drying temperature is preferably 150° C. or lower, more preferably 130° C. or lower, and still more preferably 120° C. or lower.

Drying by microwave irradiation may cause decomposition and deterioration of the hydrophilic group-containing polymer at high drying temperature, or may cause adhesion between hollow fiber membranes. Increasing temperature in excess of the hollow fiber membrane may cause decomposition and deterioration of the hydrophilic group-containing polymer, or may cause deterioration of performance of the hollow fiber membrane. Therefore, it is preferred to dry at the hollow fiber membrane temperature of 100° C. or lower, and more preferably 80° C. or lower. Although there is no particular limitation on the method for controlling the hollow fiber membrane temperature, there is a method in which microwave irradiation is performed under reduced pressure.

Since a film coefficient of material transfer can be reduced as the thickness of the hollow fiber membrane decreases, substance removing performance of the hollow fiber membrane is improved. Meanwhile, when the membrane has too small thickness, fiber breakage and drying collapse are likely to occur, which may lead to problems about production. Ease of collapse of the hollow fiber membrane has a correlation with the thickness and the inner diameter of the hollow fiber membrane. Therefore, the thickness of the hollow fiber membrane is preferably 20 μm or more, and more preferably 25 μm or more. Meanwhile, the thickness is preferably 50 μm or less, and more preferably 45 μm or less. The inner diameter of the hollow fiber membrane is preferably 80 μm or more, more preferably 100 μm or more, and still more preferably 120 μm or more, while the inner diameter is preferably 250 μm or less, more preferably 200 μm or less, and still more preferably 160 μm.

The inner diameter of the hollow fiber membrane refers to the value obtained by measuring each thickness of 16 hollow fiber membranes selected at random using lens (VH-Z100; KEYENCE. CORPORATION) at a magnification of 1,000 times of a microwatcher to determine an average “a”, followed by calculation according to equation mentioned below. The outer diameter of the hollow fiber membrane refers to the value obtained by measuring each outer diameter of 16 hollow fiber membranes selected at random using a laser displacement meter (e.g. LS5040T; KEYENCE CORPORATION).

Inner diameter (μm) of hollow fiber membrane=outer diameter (μm) of hollow fiber membrane−2×membrane thickness (μm).

The hollow fiber membrane module of the present invention is preferably obtained by building the hollow fiber membrane produced by the above method in a case.

A non-limiting example of the method for building the hollow fiber membrane into the module is shown below. First, the hollow fiber membrane is cut into the desired length, and a desired number of the cut pieces are bundled and then placed in a cylindrical case. Thereafter, both ends are temporarily capped, and a potting agent is added to both ends of the hollow fiber membrane. In this process, a method of adding a potting agent while rotating the module by means of a centrifugal machine is preferred, because the potting agent can be uniformly charged. After the potting agent is solidified, both ends are cut in such a manner that openings can be formed at both ends of the hollow fiber membrane. A header is attached to both sides of the case, and then the nozzle of the header and the case is plugged to obtain a hollow fiber membrane module.

There is a need for a hollow fiber membrane module for blood purification, such as artificial kidney, to be subjected to sterilization, and a radiation sterilization method is often used in view of low persistence and simplicity.

Therefore, since an object of the present invention is to obtain a dry-type hollow fiber membrane module, irradiation with radiation is preferably performed in a state where the water content of the hollow fiber membrane is adjusted to 10% by weight or less relative to the tare weight of the hollow fiber membrane built in the module (case). The radiation to be used may be α radiation, β radiation, γ radiation, X-ray, ultraviolet radiation, electron beam, or the like. Of these, γ radiation or electron beam is suitably used in view of low persistence and simplicity. The hydrophilic group-containing polymer incorporated into an inner surface of a hollow fiber can be fixed by causing crosslinking with a membrane material due to irradiation with radiation, which may lead to reduction in eluted substance. Therefore, irradiation with radiation is preferably performed. Low radiation dose may lead to low sterilization effect, while high radiation dose may cause decomposition of the hydrophilic group-containing polymer or the membrane material, leading to deterioration of blood compatibility. Therefore, the radiation dose is preferably 15 kGy or more, and preferably 100 kGy or less.

The permeability of the hollow fiber membrane is preferably 100 ml/hr/mmHg/m² or more, more preferably 200 ml/hr/mmHg/m² or more, and still more preferably 300 ml/hr/mmHg/m² or more. In the case of artificial kidney application, too high permeability may cause a phenomenon such as residual blood, so that the permeability is preferably 2,000 ml/hr/mmHg/m² or less, and more preferably 1,500 ml/hr/mmHg/m² or less.

### EXAMPLES

### (1) Measurement of Water Content

The mass of a hollow fiber bundle obtained by disassembling a hollow fiber membrane module was measured. The hollow fiber bundle was placed in a dryer set at 150° C. and, after drying for 3 hours, the mass was measured again. The water content of a hollow fiber was calculated by the following equation and a value, which is obtained by rounding off the second decimal position of the resulting calculated value, is used.

Water content (% by weight)=100×(a−b)/b

where
a: weight before drying (g), and b: weight after drying (g)

### (2) Measurement by X-Ray Photoelectron Spectroscopy (XPS) (Measurement of Pyrrolidone Group Content of Inner Surface of Hollow Fiber Membrane)

A hollow fiber membrane was sliced into a semi-cylindrical shape using a single-edged knife, and the measurement was performed at three points of each of a surface of the hollow fiber membrane (an inner surface of the hollow fiber membrane). The measurement sample was rinsed with ultrapure water, dried at room temperature (25° C.) at 0.5 Torr for 10 hours, and then subjected to the measurement. The following analyzer and conditions were used.

Analyzer: ESCA LAB220iXL

Excitation X-ray: monochromatic Al Kα1, 2 radiation (1486.6 eV)

X-ray diameter: 0.15 mm

Photoelectron escape angle: 90° (tilt of detector relative to sample surface)

C1s peaks are composed of five components: a component mainly derived from CHx, C—C, C═C, C—S; a component mainly derived from C—O, C—N; a component derived from π-π* satellite; a component derived from C═O; and a component derived from COO. Therefore, the peaks are deconvoluted into the five components. The COO-derived component corresponds to the peak observed at +4.0 to +4.2 eV from the main CHx or C—C peak (at about 285 eV). When calculated, the second decimal place of the peak area ratio of each component is rounded off. The ester group-derived carbon content (atomic %) was calculated by multiplying the C1s carbon content (atomic %) by the peak area ratio of the COO-derived component. As a result of peak deconvolution, a ratio of 0.4% or less is determined to be the detection limit and regarded as zero.

When the hydrophobic polymer contained in the hollow fiber membrane is polysulfone and the hydrophilic group-containing polymer has a pyrrolidone group, the vinylpyrrolidone group content of the surface of the hollow fiber membrane was calculated from the nitrogen content (c (atomic %)) and the sulfur content (d (atomic %)) according to the following equation: vinylpyrrolidone group content (% by weight) of inner surface of hollow fibermembrane=(c×111/(c×111+d×442))=100, since a molecular weight of a vinylpyrrolidone group is 111 and a molecular weight of a repeating unit constituting polysulfone is 442.

Therefore, when the hydrophilic group-containing polymer is polyvinylpyrrolidone, “vinylpyrrolidone group content (% by weight) of inner surface of the hollow fiber membrane” calculated from the above equation becomes “polyvinylpyrrolidone content (% by weight) of inner surface of hollow fiber membrane”.

### (3) Measurement by X-Ray Photoelectron Spectroscopy (XPS) (Measurement of Ester Group Content of Inner Surface of Hollow Fiber Membrane)

When using a hydrophilic group-containing polymer having an ester group, the hydrophilic group-containing polymer content of the surface of the hollow fiber membrane can be calculated using ESCA (XPS) as shown in (2). The same analyzer and conditions as in (2) were used. When the hydrophobic polymer contained in the hollow fiber membrane is polysulfone, and the hydrophilic group-containing polymer is composed of a copolymer of vinylpyrrolidone with vinyl acetate, the vinyl acetate (ester group) content of the surface was calculated from the nitrogen content (c (atomic %)), the sulfur content (d (atomic %)), and the content of carbon derived from an ester group (e (atomic %)) according to the following equation: vinyl acetate (ester group) content (% by weight) of inner surface of hollow fiber membrane=(e×86/(c×111+d×442+e×86))×100, since a molecular weight of vinylpyrrolidone is 111, a molecular weight of a repeating unit constituting polysulfone is 442, and a molecular weight of vinyl acetate is 86.

Therefore, when the hydrophilic group-containing polymer is a copolymer of vinylpyrrolidone with vinyl acetate, the content (% by weight) of a hydrophilic group-containing polymer of the inner surface of the hollow fiber membrane can be represented by the sum of “vinylpyrrolidone group content (% by weight) of the inner surface of the hollow fiber membrane” calculated in the above (2) and “vinyl acetate (ester group) content (% by weight) of the inner surface of the hollow fiber membrane” calculated by the above equation.

### (4) Measurement of Consumption Amount of Potassium Permanganate

Ultrapure water heated to 37° C. was allowed to pass through a passage (blood side passage) of the side of the liquid to be treated of the hollow fiber membrane module at a rate of 100 mL/min for 7 minutes, thereby washing the blood side passage. Subsequently, ultrapure water was allowed to pass through a passage (dialyzate side passage) of the process liquid side at a rate of 500 mL/min for 5 minutes, thereby washing a passage (dialyzate side passage) of the process liquid side. When ultrapure water was allowed to pass through a passage (blood side passage) of the side of the liquid to be treated at a rate of 100 mL/min for 3 minutes, again, 200 mL of a last part of a priming liquid flowing out during final 2 minutes was sampled and 10 mL of the a last part of a priming liquid was collected. To 10 mL of this last part of a priming liquid, 20 mL of an aqueous potassium permanganate solution (2.0×10⁻³ mol/L), 1 mL of sulfuric acid (10% by volume) and a boiling stone were added, followed by boiling for 3 minutes. The mixture was allowed to cool down for 10 minutes and then cooled to room temperature. Thereafter, the mixture was well cooled with iced water. After adding 1 mL of an aqueous 10% by weight potassium iodide solution, the mixture was well stirred and left to stand for 10 minutes, followed by titration with an aqueous sodium thiosulfate solution (1.0×10⁻² mol/L). At the time when color of the solution turns pale yellow, 0.5 mL of an aqueous 1% by weight starch solution was added, followed by well stirring at 20° C. to 30° C. After adding an aqueous sodium thiosulfate solution (1.0×10⁻² mol/L) until color of the solution turns transparent, the additive amount of the aqueous sodium thiosulfate solution was measured.

Ultrapure water, which was not allowed to pass through the hollow fiber membrane module, was also subjected to titration in the same way. The consumption amount of potassium permanganate is calculated from the amount of an aqueous sodium thiosulfate solution (f (mL)) used in titration of ultrapure water and the amount of an aqueous sodium thiosulfate solution (g (mL)) according to the following equation. An average of the results obtained by measuring twice is regarded as a measured value and a value, which is obtained by rounding off the third decimal position of the results, is used.

Consumption amount (mL) of potassium permanganate=(f−g)=h/i

where
h: factor of sodium thiosulfate, and i: factor of potassium permanganate

### (5) Trace Nitrogen Analysis

A hollow fiber membrane was freeze-crushed and the obtained freeze-crushed hollow fiber membrane was used as a measurement sample. The measurement sample was dried under reduced pressure at normal temperature (25° C.) for 2 hours and then subjected to analysis. The following analyzer and conditions were used.

Analyzer: trace nitrogen analyzer, Model ND-100 (manufactured by Mitsubishi Chemical Corporation)

Electric furnace temperature (lateral reaction furnace)

Pyrolysis section: 800° C.

Catalyst section: 900° C.

Main O₂ flow rate: 300 mL/min

O₂ flow rate: 300 mL/min

Ar flow rate: 400 mL/min

Sens: Low

An average of the results obtained by measuring three times is regarded as a measured value and has two significant figures.

### (6) Microscopic ATR Method

A hollow fiber membrane was sliced into a semi-cylindrical shape with a single-edged knife, rinsed with ultrapure water, and then dried at room temperature (25° C.) at 0.5 Torr for 10 hours. Each surface of the dried hollow fiber membrane as a sample for the measurement of a surface was measured by a microscope ATR method using IRT-3000 manufactured by JASCO Corporation. The measurement was performed in a field region (aperture) of 100 μm×100 μm within a measurement range of 3 μm×3 μm with a cumulative number of 30, and five points (lengthwise) by five points (widthwise) (25 points in total) were measured. A base line was drawn on the resulting spectrum in the wavelength range of 1,549 to 1,620 cm⁻¹, and the peak area surrounded by the base line and the positive part of the spectrum was determined to be a peak area (A_{CC}) derived from the benzene ring C═C of polysulfone. In the same way, a base line was drawn on the spectrum in the range of 1,620 to 1,711 cm⁻¹, and the peak area surrounded by the base line and the positive part of the spectrum was determined to be a peak area (A_{NCO}) derived from pyrrolidone. A base line was drawn on the spectrum in the range of 1,711 to 1,759 cm⁻¹, and the peak area surrounded by the base line and the positive part of the spectrum was determined to be a peak area (A_{COO}) derived from an ester group.

The above process was performed at three different places of the same hollow fiber. (A_{NCO})/(A_{CC}), and the average (A_{COO})/(A_{CC}) were calculated. A value, which is obtained by rounding off the third decimal position of the resulting calculated value, is used.

### (7) Method for Testing Deposition of Human Platelets

A double-side tape was bonded to an 18 mmφ polystyrene circular plate, and the hollow fiber membrane was fixed thereon. The attached hollow fiber membrane was sliced into a semi-cylindrical shape with a single-edged knife so that the inner surface of the hollow fiber membrane was exposed. It should be carefully performed, because if there is dirt, a scratch, a fold, or the like on the inner surface of the hollow fiber, platelets may be deposited on such a portion so that the evaluation may not be correctly performed. The circular plate was attached to a cylindrical cut piece of Falcon (registered trademark) tube (No. 2051, 18 mmφ, 3 cm in length) so that the hollow fiber membrane-carrying surface was placed inside the cylinder, and the gap was filled with Parafilm. The interior of the cylindrical tube was washed with a saline solution and then filled with a saline solution. Heparin was added at a concentration of 50 U/mL to healthy human venous blood (number of red blood cells: 4,500,000 to 5,000,000 cells/mm³, number of white blood cells: 5,000 to 8,000 cells/mm³, platelets: 200,000 to 500,000 platelets/mm³) immediately after the blood sampling. After the saline solution was discharged from the cylindrical tube, 1.0 mL of the blood was placed in the cylindrical tube within 30 minutes after the sampling and shaken at 700 rpm at 37° C. for 1 hour. Thereafter, the hollow fiber membrane was washed with 10 mL of a saline solution and 1 mL of a 2.5% by weight glutaraldehyde saline solution was added, and then the blood component was fixed thereon by being left to stand. After a lapse of one or more hours, the blood component was washed with 20 mL of distilled water. The washed hollow fiber membrane was dried at normal temperature (25° C.) under a reduced pressure of 0.5 Torr for 10 hours. The hollow fiber membrane was then bonded to the sample stage of a scanning electron microscope with a double-side tape. A Pt—Pd thin film was then formed on the surface of the hollow fiber membrane by sputtering, so that a sample was obtained. The inner surface of the hollow fiber membrane sample was observed with a field emission-type scanning electron microscope (S800 manufactured by Hitachi, Ltd.) at a magnification of 1,500 times, and the number of the deposited platelets per field (4.3×10³ μm²) was counted. The number of the deposited platelets (platelets/4.3×10³ μm²) was defined as the average (obtained by rounding off the second decimal position) of the numbers of the deposited platelets which were counted in ten different fields at and around the longitudinal center of the hollow fiber. When the number of the deposited platelets exceeds 50 platelets/4.3×10³ μm² per field, it was counted as 50 platelets. The longitudinal ends of the hollow fiber were omitted from the objects to be measured for the number of deposits, because blood tended to stay thereon.

### (8) Content (% by Weight) of Hydrophilic Group-Containing Polymer of Outer Surface of Hollow Fiber Membrane

In the same manner as in the above (2) and (3), except that an outer surface of a hollow fiber membrane is selected as the objective surface to be measured, the content (% by weight) of a hydrophilic group-containing polymer of an outer surface of a hollow fiber membrane was determined.

### Example 1

Sixteen percentage (16%) by weight of polysul-fone (manufactured by Amoco Corporation, “Udel” P-3500 LCD MB7, molecular weight of 77,000 to 83,000), 4% by weight of polyvinylpyrrolidone (K30, manufactured by International. Specialty Products, Inc.; hereinafter abbreviated to ISP), and 2% by weight of polyvinylpyrrolidone (K90, manufactured by ISP) were dissolved with heating in 77% by weight of N,N-dimethylacetamide and 1% by weight of water to obtain a membrane forming stock solution.

In a solution of 66% by weight of N,N-dimethylacetamide and 33.97% by weight of water, 0.03% by weight of a vinylpyrrolidone/vinyl acetate (6/4 (molar ratio)) random copolymer (“KOLLIDON” (registered trademark) VA64″, manufactured by BASF Corporation) was dissolved to obtain an injection liquid.

The membrane forming stock solution was fed to a spinning spinneret at a temperature of 50° C., and discharged through an outside tube of an orifice-type double annulation spinneret with a circular slit part having an outer diameter of 0.35 mm and an inner diameter of 0.25 mm, while the injection liquid was discharged through an inside tube. The discharged membrane forming stock solution was allowed to pass through a 350 mm dry-zone atmosphere at a temperature of 30° C. and a dew point of 28° C. and to pass through a coagulation bath of 100% by weight of water at a temperature of 40° C. The hollow fiber membrane was allowed to pass through a water washing step at 60 to 75° C. for 90 seconds, a drying step at 130° C. for 2 minutes, and a crimping step at 160° C., and then the resulting hollow fiber membrane was wound into a bundle. The hollow fiber membrane had an inner diameter of 200 μm and an outer diameter of 280 μm. The hollow fiber membrane was housed in a case so as to have an inner surface area of 1.5 m², and both ends of the hollow fiber membrane were fixed onto the ends of the case with a potting material. The ends of the potting material were partially cut such that openings were formed at both ends of the hollow fiber membrane, and a header was attached to both sides of the case to obtain a module including a built-in hollow fiber membrane. Thereafter, the air in the module was replaced by nitrogen, followed by irradiation with γ radiation in a radiation dose of 25 kGy to obtain a hollow fiber membrane module 1. The water content of the resulting hollow fiber membrane module, the consumption amount of potassium permanganate, the hydrophilic group-containing polymer contents of the inner and outer surfaces of the hollow fiber membrane, the microscopic ATR of the inner surface, and the number of deposited platelets were measured. The results are shown in Table 1. The hollow fiber membrane module thus obtained is that in which the hydrophilic group-containing polymer uniformly exists on the inner surface hollow fiber and fewer platelets are deposited, and little eluted substance is euluted even though irradiation with γ radiation was performed under the condition of low water content.

### Example 2

In the same manner as in Example, except that the amount of the hydrophilic group-containing polymer to be added to the injection liquid was adjusted to 0.01% by weight and the content of the water was adjusted to 33.99% by weight, a hollow fiber membrane was formed and then built in a case to obtain a hollow fiber membrane module 2. The results are shown in Table 1. The hollow fiber membrane module thus obtained is that in which the hydrophilic group-containing polymer uniformly exists in the hollow fiber membrane and fewer platelets are deposited, and little eluted substance is eluted.

### Example 3

In the same manner as in Example 1, except that a vinylpyrrolidone/vinyl acetate (7/3 (molar ratio)) copolymer (“Luviskol VA73”, manufactured by BASF Corporation) was used as the hydrophilic group-containing polymer to be added to the injection liquid, a hollow fiber membrane was formed and then built in a case to obtain a hollow fiber membrane module 3. The results are shown in Table 1. In the same way as Example 1, a hollow fiber membrane module, which elutes little eluted substance, was obtained.

### Example 4

In the same manner as in Example 1, except that a vinylpyrrolidone/vinyl acetate (3/7 (molar ratio)) copolymer (“Luviskol VA37”, manufactured by BASF Corporation) was used as the hydrophilic group-containing polymer to be added to the injection liquid, a hollow fiber membrane was formed and then built in a case to obtain a hollow fiber membrane module 4. The results are shown in Table 1. In the same way as Example 1, a hollow fiber membrane module, which elutes little eluted substance, was obtained.

### Example 5

Under the same conditions as in Example 1, except that the hydrophilic group-containing polymer was not added to the injection liquid, a hollow fiber membrane was formed and then built in a case to obtain a hollow fiber membrane module.

Then, an aqueous solution of 0.01% by weight of a vinylpyrrolidone/vinyl acetate (6/4 (molar ratio)) random copolymer (“KOLLIDON” (registered trademark) VA64″, manufactured by BASF Corporation) at 80° C. was allowed to pass from an inlet (15A) of the liquid to be treated of the hollow fiber membrane module to an outlet (15B) of the liquid to be treated at a rate of 500 mL/min for one minute passed (at this time, the inlet (15A) of the liquid to be treated and the outlet (15B) of the liquid to be treated are opened, while the inlet (16A) of the process liquid and the outlet (16B) of the process liquid are closed).

Subsequently, the solution was allowed to pass from the inlet (15A) of the liquid to be treated to the inlet (16A) of the process liquid at a rate of 500 mL/min for one minute passed (at this time, the inlet (15A) of the liquid to be treated and the inlet (16A) of the process liquid are opened, but the outlet (15B) of the liquid to be treated and the outlet (16B) of the process liquid are closed).

Subsequently, the filling liquid was pressed from the outer surface of the hollow fiber membrane side to the inner surface of the hollow fiber membrane side with compressed air at 100 kPa (at this time, the inlet (16A) of the process liquid and the inlet (15A) of the liquid to be treated are opened, while the outlet (15B) of the liquid to be treated and the outlet (16B) of the process liquid are closed).

In a state where the pressure applied to the outer surface of the hollow fiber membrane side is maintained at 100 kPa, compressed air was fed to a direction of from the outlet (15B) side of the liquid to be treated to the inlet (15A) side of the liquid to be treated, and a liquid inside the hollow fiber membrane was pressed to the inlet (15A) side of the liquid to be treated (at this time, the outlet (15B) of the liquid to be treated and the inlet (15A) of the liquid to be treated are opened, while the inlet (16A) of the process liquid and the outlet (16B) of the process liquid are closed), leading to a state where only the hollow fiber membrane is wetted.

Furthermore, the hollow fiber was dried by irradiating this module with microwave (6 kW) and the air in the module was replaced by nitrogen, followed by irradiation with γ radiation in a radiation dose of 25 kGy to obtain a hollow fiber membrane module 4. The results are shown in Table 1. The hollow fiber membrane module thus obtained is that in which the hydrophilic group-containing polymer uniformly exists in the hollow fiber membrane and fewer platelets are deposited, and little eluted substance is eluted.

### Comparative Example 1

In the same manner as in Example 1, except that 18% by weight of polysulfone (“Udel” P-3500, manufactured by Amoco Corporation), 6% by weight of polyvinylpyrrolidone (K30, manufactured by International Specialty Products, Inc.; hereinafter abbreviated to ISP), and 3% by weight of polyvinylpyrrolidone (K90, manufactured by ISP) were dissolved with heating in 72% by weight of N,N-dimethylacetamide and 1% by weight of water to obtain a membrane forming stock solution, and that the hydrophilic group-containing polymer was not added to the injection liquid, a hollow fiber membrane was formed and then built in a case to obtain a hollow fiber membrane module 5. The results are as shown in Table 1. Because of large polyvinylpyrrolidone content in the hollow fiber membrane regardless of sufficient hydrophilic group-containing polymer content of the inner surface, numerous eluted substance was observed.

### Comparative Example 2

Under the same conditions as in Example 1, except that the hydrophilic group-containing polymer was not added to the injection liquid, a hollow fiber membrane was formed and then built in a case to obtain a hollow fiber membrane module. Then, an aqueous solution of 0.1% by weight of a vinylpyrrolidone/vinyl acetate (6/4 (molar ratio)) random copolymer (“KOLLIDON” (registered trademark) VA64″, manufactured by BASF Corporation) was allowed to pass from the blood side inlet of the hollow fiber membrane module to the outlet at a rate of 500 mL/min for one minute, and to pass from blood side inlet to the dialyzate side outlet at a rate of 500 mL/min for one minute passed. Then, the filling liquid was pressed from the dialyzate side to the blood side with compressed air at 100 kPa. Thereafter, the filling liquid on the blood side was blown so that the aqueous solution was held only in the hollow fiber membrane. In other words, a state where only the hollow fiber membrane is wetted was achieved in the same manner as in Example 5.

Furthermore, the module was dried in a reduced-pressure dryer at a normal temperature (25° C.). Thereafter, the air in the module was replaced by nitrogen, followed by irradiation with γ radiation in a radiation dose of 25 kGy to obtain a hollow fiber membrane module 6. The water content of the resulting hollow fiber membrane module 6, the consumption amount of potassium permanganate, the hydrophilic group-containing polymer contents of the inner and outer surfaces of the hollow fiber membrane, the microscopic ATR of the inner surface, and the number of deposited platelets were measured. The results are shown in Table 1. When coated with the hydrophilic group-containing polymer after forming a membrane, the module has high hydrophilicity and is excellent in suppression of platelet deposition, but numerous eluted substance was observed.

### Comparative Example 3

Under the same conditions as in Example 1, except that a solution prepared by dissolving 10% by weight of a vinylpyrrolidone/vinyl acetate (6/4 (molar ratio)) random copolymer (“KOLLIDON” (registered trademark) VA64″, manufactured by BASF Corporation) was used as the injection liquid, a hollow fiber membrane was formed and then built in a case to obtain a hollow fiber membrane module, followed by irradiation with γ radiation. The water content of the resulting hollow fiber membrane module 7, the consumption amount of potassium permanganate, the hydrophilic group-containing polymer contents of the inner and outer surfaces of the hollow fiber membrane, the microscopic ATR of the inner surface, and the number of deposited platelets were measured. The results are shown in Table 1. The module has high hydrophilicity, but is slightly inferior in platelet deposition inhibitory effect and numerous eluted substance was observed.

### Comparative Example 4

Under the same conditions as in Comparative Example 1, except that 18% by weight of polysulfone (“Udel” P-3500, manufactured by Amoco Corporation) and 9% by weight of a vinylpyrrolidone/vinyl acetate (6/4 (molar ratio)) random copolymer (“KOLLIDON” (registered trademark) VA64, manufactured by BASF Corporation) were dissolved with heating in a mixed solvent of 72% by weight of N,N′-dimethylacetamide and 1% by weight of water to obtain a solution and the resulting solution was used as the membrane forming stock solution, a hollow fiber membrane was formed and then built in a case to obtain a hollow fiber membrane module, followed by irradiation with γ radiation. The water content of the resulting hollow fiber membrane module 8, the consumption amount of potassium permanganate, the hydrophilic group-containing polymer contents of the inner and outer surfaces of the hollow fiber membrane, the microscopic ATR of the inner surface, and the number of deposited platelets were measured. The module is excellent in platelet deposition inhibitory effect, but numerous eluted substance was observed.

TABLE 1

| | Production conditions | | | |
|---|---|---|---|---|
| | Hydrophilic | Hydrophilic | Amount added | |
| | group-containing | group-containing | to injection | Hydrophilic group-containing |
| | polymer added to | polymer added to | liquid (% by | polymer added after formation |
| | stock solution | injection liquid | weight) | of membrane |
| Example 1 | PVP | VA64 | 0.03 | No addition |
| Example 2 | PVP | VA64 | 0.01 | No addition |
| Example 3 | PVP | VA73 | 0.05 | No addition |
| Example 4 | PVP | VA37 | 0.03 | No addition |
| Example 5 | PVP | No addition | — | VA64 |
| | | | | (100 ppm aqueous solution) |
| Comparative | PVP | No addition | — | No addition |
| Example 1 | | | | |
| Comparative | PVP | No addition | — | VA64 |
| Example 2 | | | | (1,000 ppm aqueous solution) |
| Comparative | PVP | VA64 | 10 | No addition |
| Example 3 | | | | |
| Comparative | VA64 | No addition | — | No addition |
| Example 4 | | | | |

TABLE 2

| | Hollow fiber membrane module | | | | |
|---|---|---|---|---|---|
| | | Content of | | | Content of |
| | | hydrophilic | Content of | Content of | hydrophilic |
| | Water | group-containing | vinylpyrrolidone | vinyl acetate | group-containing |
| | content of | polymer in inner | in inner surface | in inner surface | polymer in outer |
| | hollow fiber | surface of hollow | of hollow fiber | of hollow fiber | surface of hollow |
| | membrane | fiber membrane | membrane | membrane | fiber membrane |
| | (% by weight) | (% by weight) | (% by weight) | (% by weight) | (% by weight) |
| Example 1 | 0.34 | 40.8 | 31.6 | 9.2 | 31.3 |
| Example 2 | 0.44 | 29.6 | 23.8 | 5.8 | 30.5 |
| Example 3 | 0.58 | 37.2 | 30.5 | 6.7 | 31.8 |
| Example 4 | 0.42 | 35.2 | 24.5 | 10.7 | 32.1 |
| Example 5 | 0.73 | 33.4 | 24.5 | 8.9 | 31.5 |
| Comparative | 0.41 | 47.4 | 47.4 | — | 53.2 |
| Example 1 | | | | | |
| Comparative | 0.50 | 38.1 | 27.1 | 11.0 | 39.8 |
| Example 2 | | | | | |
| Comparative | 0.45 | 48.6 | 33.2 | 15.4 | 38.3 |
| Example 3 | | | | | |
| Comparative | 0.42 | 37.3 | 22.4 | 14.9 | 25.5 |
| Example 4 | | | | | |

| | Hollow fiber membrane module | | | | |
|---|---|---|---|---|---|
| | | | ATR of | ATR of | Number of |
| | Consumption | Nitrogen | inner surface | inner surface | deposited |
| | amount of | content in | of hollow fiber | of hollow fiber | platelets |
| | potassium | hollow fiber | membrane | membrane | (platelets/ |
| | permanganate | membrane | (A_{NCO})/ | (A_{COO})/ | 4.3 × |
| | (mL/m²) | (% by weight) | (A_{CC}) | (A_{CC}) | 10³ μm²) |
| Example 1 | 0.13 | 0.20 | 0.62 | 0.06 | 1.2 |
| Example 2 | 0.12 | 0.19 | 0.6 | 0.02 | 5.1 |
| Example 3 | 0.18 | 0.22 | 0.68 | 0.01 | 9.2 |
| Example 4 | 0.16 | 0.18 | 0.55 | 0.07 | 8.1 |
| Example 5 | 0.09 | 0.21 | 0.76 | 0.09 | 1.5 |
| Comparative | 0.31 | 0.60 | 0.87 | — | 50 |
| Example 1 | | | | | |
| Comparative | 0.27 | 0.23 | 0.75 | 0.09 | 2.1 |
| Example 2 | | | | | |
| Comparative | 0.55 | 0.25 | 1.1 | 0.14 | 5 |
| Example 3 | | | | | |
| Comparative | 0.35 | 0.045 | 0.44 | 0.22 | 0.8 |
| Example 4 | | | | | |

### REFERENCE SIGNS LIST

11: Cylindrical case

13: Hollow fiber membrane

14A: Header

14B: Header

15A: Inlet of liquid to be treated

15B: Outlet of liquid to be treated

16A: Nozzle (inlet of process liquid)

16B: Nozzle (outlet of process liquid)

17: Partition wall

1. A hollow fiber membrane module comprising a built-in hollow fiber membrane including a hydrophobic polymer and a hydrophilic group-containing polymer, the hollow fiber membrane module satisfying the following items:
(a) the water content of the hollow fiber membrane is 10% by weight or less relative to the tare weight of the hollow fiber membrane,
(b) the hydrophobic polymer contains no nitrogen, the hydrophilic group-containing polymer contains nitrogen, and the nitrogen content of the hollow fiber membrane is 0.05% by weight or more and 0.4% by weight or less,
(c) the content of the hydrophilic group-containing polymer in the inner surface of the membrane is 20% by weight or more and 45% by weight or less, and
(d) the consumption amount of an aqueous potassium permanganate solution (2.0×10⁻³ mol/L) used for titrating an eluted substance in 10 mL of a last part of a priming liquid is 0.2 mL or less per 1 m² of a membrane area.

2. The hollow fiber membrane module according to claim 1, wherein the number of deposited human platelets in the inner surface of the hollow fiber membrane is 20 platelets/(4.3×10³ μm²) or less.

3. The hollow fiber membrane module according to claim 1, wherein the hydrophilic group-containing polymer has a pyrrolidone group.

4. The hollow fiber membrane module according to claim 1, wherein the hydrophilic group-containing polymer has an ester group.

5. The hollow fiber membrane module according to claim 4, wherein the ester group is derived from at least one selected from a vinyl carboxylic acid ester, an acrylic acid ester, and a methacrylic acid ester.

6. The hollow fiber membrane module according to claim 3, wherein the hydrophilic group-containing polymer is a copolymer of vinyl acetate with vinylpyrrolidone.

7. The hollow fiber membrane module according to claim 1, wherein the hydrophobic polymer is a polysulfone-based polymer.

8. A method for producing a hollow fiber membrane used in the hollow fiber membrane module according to claim 1, the method comprising a step of using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.

9. A method for producing a hollow fiber membrane, the method comprising using a solution which contains a hydrophobic polymer containing no nitrogen as a membrane forming stock solution, using a solution which contains 0.01% by weight or more and 1% by weight or less of a hydrophilic group-containing polymer containing nitrogen as an injection liquid, and discharging the solutions through a double annulation spinneret.

10. The method for producing a hollow fiber membrane according to claim 8, wherein the hydrophilic group of the hydrophilic group-containing polymer includes a pyrrolidone group.

11. The method for producing a hollow fiber membrane according to claim 8, wherein the hydrophilic group-containing polymer has an ester group.

12. The method for producing a hollow fiber membrane according to claim 11, wherein the ester group is derived from at least one selected from a vinyl carboxylic acid ester, an acrylic acid ester, and a methacrylic acid ester.

13. The method for producing a hollow fiber membrane according to claim 10, wherein the hydrophilic group-containing polymer is a copolymer of vinyl acetate with vinylpyrrolidone.

14. The method for producing a hollow fiber membrane according to claim 8, wherein the hydrophobic polymer is a polysulfone-based polymer.

15. A method for producing a hollow fiber membrane module, the method comprising building the hollow fiber membrane produced by the method according to claim 8 in a case.

16. The method for producing a hollow fiber membrane module according to claim 15, wherein irradiation with radiation is performed in a state where the water content of the hollow fiber membrane is adjusted to 10% by weight or less relative to the tare weight of the hollow fiber membrane built in the module.
